(19) United States
(12) Patent Application Publication
(10) Pub. No.: US 2008/0287405 A1
(43) Pub. Date: Nov. 20, 2008

(54) Compositions and Methods Relating to Protein Kinase Inhibitors

(76) Inventor: Victor J. Thannickal, Ann Arbor, MI (US)

Correspondence Address: Casimir Jones, S.C., 440 Science Drive, Suite 203, Madison, WI, 53711, US

(21) Appl. No.: 11/596,523

(22) PCT Filed: May 16, 2005
(86) PCT No.: PCT/US05/17131

§ 371 Date: Oct. 19, 2007

Related U.S. Application Data
(60) Provisional application No. 60/571,385, filed on May 14, 2004.

(51) Int. Cl. A61K 31/519; A61K 31/56; A61P 11/00

(52) U.S. Cl. 514/171; 514/262.1

(57) ABSTRACT

The present invention relates to compounds useful for treating fibrotic disorders, methods for their discovery, and their therapeutic use. In particular, the present invention provides pyrazolopyrimidine compounds and related compounds and methods of using pyrazolopyrimidine derivatives and related compounds as therapeutic agents to treat a number of conditions associated with fibrotic disorders.

This patent application claims priority to U.S. Provisional Patent Application No. 60/571,385, filed May 14, 2004.

This invention was supported in part with NIH grant R01HL67967. The United States government may have rights in this invention.

### FIELD OF THE INVENTION

The present invention relates to compounds useful for treating fibrotic disorders, methods for their discovery, and their therapeutic use. In particular, the present invention provides pyrazolopyrimidine compounds and related compounds and methods of using pyrazolopyrimidine derivatives and related compounds as therapeutic agents to treat a number of conditions associated with fibrotic disorders.

### BACKGROUND OF THE INVENTION

Inflammation and repair are stereotypical host responses to injury of adult mammalian tissues. Dysregulation of either of these processes may lead to pathological outcomes resulting in varying degrees of chronic inflammation and fibrosis (see, e.g., Nathan, C. Nature 420:846-852 (2002); herein incorporated by reference in its entirety). Regulatory mechanisms of inflammatory responses are better understood than are tissue repair/regenerative responses. Epithelial/endothelial regeneration to restore barrier functions and maintain tissue architecture is facilitated by connective tissue cells. Fibroblasts, in particular, represent a versatile and phenotypically heterogeneous population of connective tissue cells that transiently appear in response to injury and normally disappear following repair (see, e.g., Singer, A. J., and Clark, R. A. Cutaneous wound healing. N Engl J Med 341:738-746 (1999); herein incorporated by reference in its entirety). Recruitment of fibroblasts to sites of tissue injury likely occurs in diverse organ systems in which epithelial/endothelial cells and the underlying basement membrane(s) are disrupted (see, e.g., Svee, K., et al., J Clin Invest 98:1713-1727 (1996); herein incorporated by reference in its entirety). Elaboration of a provisional matrix by fibroblasts is an early “repair” function that aids epithelial/epidermal cell migration (see, e.g., Clark, R. A., et al., J Invest Dermatol 79:264-269 (1982); herein incorporated by reference in its entirety). Phenotypic transition of fibroblasts to myofibroblasts further facilitates re-epithelialization by contracting wounds and bringing epithelial margins into closer apposition (see, e.g., Singer, A. J., and Clark, R. A., N Engl J Med 341:738-746 (1999); herein incorporated by reference in its entirety). Resolution of the repair response is associated with successful re-epithelialization and the eventual removal of myofibroblasts by a mechanism that likely involves apoptosis (see, e.g., Desmouliere, A., et al., Am J Pathol 146:56-66 (1995); herein incorporated by reference in its entirety). The persistence of myofibroblasts at sites of tissue injury is a consistent finding in most, if not all, human fibrotic diseases (see, e.g., Tomasek, J. J., et al., Nat Rev Mol Cell Biol 3:349-363 (2002); herein incorporated by reference in its entirety).

Transforming growth factor-β1 (TGF-β1) is a multifunctional cytokine that plays a central role in fibrotic diseases (see, e.g., Border, W. A., et al., J Clin Invest 90:1-7 (1992); Border, W. A., and Noble, N. A., J Clin Invest 96:655-656 (1995); each herein incorporated by reference in their entireties). TGF-β1 induces myofibroblast differentiation both in vitro (see, e.g., Desmouliere, A., et al., J Cell Biol 122:103-111 (1993); herein incorporated by reference in its entirety) and in vivo (see, e.g., Sime, P. J., et al., J Clin Invest 100:768-776 (1997); herein incorporated by reference in its entirety). Recent studies demonstrate that this phenotypic transition is critically dependent on cell adhesive events and integrin signaling via focal adhesion kinase (FAK) (see, e.g., Thannickal, V. J., et al., J Biol Chem 278:12384-12389 (2003); herein incorporated by reference in its entirety). In addition to inducing differentiation, there is evidence that TGF-β1 may also inhibit myofibroblast apoptosis (see, e.g., Zhang, H. Y., and Phan, S. H., Am J Respir Cell Mol Biol 21:658-665 (1999); Jelaska, A., and Kom, J. H., Arthritis Rheum 43:2230-2239 (2000); each herein incorporated by reference in their entireties), although mechanisms for this effect are unclear. It was recently demonstrated that activation of the phosphatidylinositol 3-kinases (PI3K)-protein kinase B (PKB/Akt) pathway by TGF-β1 was, at least in part, necessary for its growth and anti-apoptotic effects (see, e.g., Kim, G., et al., Arthritis Rheum 46:1504-1511 (2002); herein incorporated by reference in its entirety).

The PI3K-Akt pathway is known to regulate a number of cellular processes including cell cycle progression, glucose metabolism, angiogenesis, cell motility and survival (see, e.g., Cantley, L. C., Science 296:1655-1657 (2000); herein incorporated by reference in its entirety). Phosphorylation and activation of PKB/Akt plays a central role in cell survival/anti-apoptotic signaling by targeting multiple substrates (see, e.g., Datta, S. R., et al., Genes Dev 13:2905-2927 (1999); herein incorporated by reference in its entirety).

Idiopathic pulmonary fibrosis (IPF) is a progressive, fibrosing disease of the distal alveolar air spaces of the lung that culminates in death, usually within 3-5 years (see, e.g., American Thoracic Society, Am J Respir Crit. Care Med 161:646-664 (2000); herein incorporated by reference in its entirety). There is currently no effective therapy for IPF. Anti-inflammatory agents and potent immunosuppressive regimens have shown no benefit. Mortality in IPF is increased in patients with greater profusion of so-called “fibroblastic foci” on lung histopatholgy (see, e.g., King, T. E., et al., Am J Respir Crit. Care Med 164:1025-1032 (2001); Flaherty, K. R., et al., Am J Respir Crit. Care Med 167:1410-1415 (2003); each herein incorporated by reference in their entireties). The biological mechanism(s) responsible for the activation and persistence of fibroblasts/myofibroblasts in fibroblastic foci of IPF are unclear. Previous studies have demonstrated that fibroblasts acquire apparently stable phenotypes when cultured in vitro after exposure to in vivo inflammatory/fibrotic conditions (see, e.g., Korn, J. H., J Clin Invest 71:1240-1246 (1983); Duncan, M. R., and Berman, B., J Clin Invest 79:1318-1324 (1987); Ramos, C., et al., Am J. Respir Cell Mol Biol 24:591-598 (2001); each herein incorporated by reference in their entireties). Such sustained phenotypic alterations include changes in growth characteristics, collagen production and metabolic functions. Whether similar stable alterations occur in the signaling program of cells cultured ex vivo and, if so, whether such changes reflect in vivo cell phenotype/behavior that can be modulated by anti-fibrotic therapy are not known.

Protein kinase inhibitors (PKIs) have recently been shown to be effective modulators of cell phenotype by targeting signal transduction pathways in human cancers (see, e.g., Dancey, J., and Sausville, E. A., Nat Rev Drug Discov 2:296-313 (2003); herein incorporated by reference in its entirety). There has been increasing interest in PKIs for certain non-oncologic diseases, primarily to treat inflammatory conditions such as rheumatoid arthritis (see, e.g., Cohen, P., Nat Rev Drug Discov 1:309-315 (2002); herein incorporated by reference in its entirety). As in rheumatoid arthritis, PKIs that target p38 MAPK appears to be effective in animal models of lung injury and fibrosis primarily by blocking inflammatory pathways (see, e.g., Underwood, D. C., et al., Am J Physiol Lung Cell Mol Physiol 279:L895-902 (2000); Matsuoka, H., et al., Am J Physiol Lung Cell Mol Physiol 283:L103-112 (2002); each herein incorporated by reference in their entireties). Potential efficacy and mechanisms of action of PKIs to specifically target fibrotic responses in vivo are not known.

What is needed is an improved understanding of the mechanisms surrounding fibrous diseases. What is also needed are improved methods of treating fibrous diseases.

### SUMMARY

The present invention relates to compounds useful for treating fibrotic disorders, methods for their discovery, and their therapeutic use. In particular, the present invention provides pyrazolopyrimidine compounds and related compounds and methods of using pyrazolopyrimidine derivatives and related compounds as therapeutic agents to treat a number of conditions associated with fibrotic disorders.

In certain embodiments, the present invention provides a method of treating a fibrotic disorder. In such embodiments, the method provides a subject having a fibrous disease and an effective amount of at least one pyrazolopyrimidine compound. In further embodiments, the effective amount of at least one pyrazolopyrimidine compound is administered to the subject. In additional embodiments, the fibrous disease is characteristic by activated PKB/Akt. In some embodiments, the subject has been diagnosed to have elevated levels of activated PKB/Akt. In even further embodiments, the administering attenuates a fibrotic response. In further embodiments, the fibrotic response results in fibrotic pulmonary lesions. In other embodiments, the fibrotic pulmonary lesions comprise idiopathic pulmonary fibrosis (IPF). In yet other embodiments, the at least one pyrazolopyrimidine compound comprises 4-amino-5-(4-chlorophenyl)-7-(t-butyl)pyrazolo[3,4-d]pyrimidine.

In certain embodiments, the present invention provides a therapeutic composition comprising 4-amino-5-(4-chlorophenyl)-7-(t-butyl)pyrazolo[3,4-d]pyrimidine and a therapeutic excipient. In such embodiments, the 4-amino-5-(4-chlorophenyl)-7-(t-butyl)pyrazolo[3,4-d]pyrimidine and the therapeutic excipient treats fibrous diseases. In further embodiments, the therapeutic composition further comprises one or more compounds that treat fibrous disease, and related conditions or symptoms. In even further embodiments, the fibrosis disease comprises idiopathic pulmonary fibrosis (IPF).

### DESCRIPTION OF THE FIGURES

FIG. 1 shows constitutive and TGF-β1-induced PKB/Akt and FAK phosphorylation in fibroblasts isolated from lungs of patients with idiopathic pulmonary fibrosis (IPF: F1, F2, F3) and unaffected, normal-appearing regions of lung from patients undergoing resection for lung cancer (normal: N1, N2, N3). A: Cells were grown to near-confluence in the presence of 10% FBS, serum-deprived for 24 h and treated with/without TGF-β1 (2 ng/ml×16 h). Cell lysates were then obtained in RIPA buffer, subjected to SDS-PAGE and immunoblotted with specific antibodies against phospho-S⁴⁷³ Akt and phospho-Y³⁹⁷ FAK; blots were stripped and re-probed for total Akt and FAK, respectively. B: Densitometric analyses of the ratio of phosphorylated:total Akt and FAK for each of the bands represented in (A) was determined. Bars in graphs represent mean ±S.E.M., n=3 for each set of samples.

FIG. 2 shows in vitro effects of the protein kinase inhibitor, AG1879, on constitutive PKB/Akt and FAK phosphorylation in fibroblasts isolated from a representative patient with IPF (A) and TGF-β1-induced phosphorylation of these protein kinases in normal fibroblasts (B). IPF fibroblasts were serum-deprived for 24 h and exposed to varying doses of AG1879 (AG, indicated doses) and an inactive analog of AG1879 (c/AG, 10 μM) for 16 h and cell lysates obtained in RIPA buffer. Normal fibroblasts were treated with TGF-β1 (2 ng/ml) in the presence/absence of active AG1879 or inactive analog at the doses for 16 h prior to cell lysis. Cell lysates were then subjected to SDS-PAGE and immunoblotted with specific antibodies against phospho-S⁴⁷³ Akt and phospho-Y³⁹⁷ FAK; blots were stripped and re-probed for total Akt and FAK, respectively.

FIG. 3 shows immunohistochemical analyses of formalin-fixed, paraffin-embedded lung tissue from mice injured with intra-tracheal bleomycin and receiving active AG1879-PKI or control analog drug. Staining is with a monoclonal antibody against α-SMA and phospho-specific antibodies against PKB/Akt and FAK.

FIG. 4 shows modulation of lung fibroblast signaling/phenotype in bleomycin-injured mice treated with the PKI, AG1879. C57BL/6J mice were given intratracheal (IT) saline or bleomycin on day 1. Bleomycin-injured mice were given intraperitoneal (IP) injections of saline, active AG1879, or an inactive analog of AG1879 starting on day 8. Fibroblasts were isolated from lung explants on day 14 and expanded in in vitro cell culture. Cells were lysed in RIPA buffer at passage 2 and 90% confluency. Cell lysates were subjected to sodium dodecyl sulfate polyacrylamide gel electrophoresis and immunoblotted with antibodies against α-SMA (a marker of myofibroblast differentiation), phospho-S⁴⁷³ Akt, and phospho-Y³⁹⁷ FAK.

FIG. 5 shows in vivo effects of systemically administered AG1879 on fibrotic responses in lungs of mice subjected to bleomycin-induced injury. Mice were given intratracheal bleomycin (BL) or saline (control). BL-injured mice received no intervention (BL), intraperitoneal injection with an inactive analog of AG1879 (BL+AG/inactive), or active AG1879 (BL+AG1879) starting on the day of initial injury (day 1) or a week following initial injury (day 8). A) Lungs were harvested on day 14 following bleomycin injury and total lung collagen determined as described in “Methods”. Value are expressed as mean ±S.E.M., n=6 per group. *p<0.05 compared to bleomycin alone or bleomycin +AG/inactive. This is one of three separate experiments that demonstrated similar results. B) Representative histopathology (hematoxylin and eosin staining, top panels; Masson's trichrome blue staining for collagen, bottom panels) of the lungs of mice intra-tracheally instilled with saline (control) or bleomycin (BL). Bleomycin-injured mice were administered intraperitoneal injections of inactive drug analog (BL+AG/inactive) or the active protein kinase inhibitor, AG1879 (BL+AG1879), on the day of initial injury. Lungs were examined at day 14 following bleomycin injury.

FIG. 6 shows the effect of AG1879 on the inflammatory response in the lungs of bleomycin-injured mice. Mice given intra-tracheal saline (control) or bleomycin (BL) were administered either inactive drug analog (BL+AG/inactive) or active protein kinase inhibitor (BL+AG1879) on the day of initial injury. Collagenase digests of lung were performed on day 7 and differential counts made of subpopulations of monocytes/macrophages, lymphocytes and neutrophils. Values represent mean ±S.E.M., n=6 per group.

FIG. 7 shows modulation of lung fibroblast signaling/phenotype in bleomycin-injured mice treated with the protein kinase inhibitor, AG1879. Mice given intra-tracheal saline (control) or bleomycin (BL) were administered either inactive drug analog (BL+AG/inactive) or active protein kinase inhibitor (BL+AG1879) on the day of initial injury. Fibroblasts were isolated from lung explants on day 14 and expanded in in vitro cell culture, initially in the presence of 10% fetal bovine serum and then in the absence of serum for 24 h. Cell lysates were obtained and subjected to SDS-PAGE and immunoblotted with antibodies against the proteins indicated. Blots are representative of three separate experiments that demonstrated similar results.

FIG. 8 shows in vivo effects of AG1879 on fibroblast phenotype. Fibroblasts isolated from mice receiving active/inactive drug in vivo were not treated with drugs in cultured fibroblasts. Fibroblasts were lysed and immunoblotted for phospho-specific antibodies against FAK and PKB/Akt.

FIG. 9 shows in vivo effects of Gleevec on lung fibroblast myofibroblast phenotype in bleomycin-injured mice. Mice were treated with Gleevec by IP injection (6 mg/kg) or oral gavage (6 mg/kg) for 21 days. Fibroblasts were isolated from lung explants and cell lysates subjected to SDS-PAGE and immunoblotted for α-SMA. Blots were stripped and re-probed for α-tubulin. Additionally, the effects of Gleevec on pro-fibrotic signaling of fibroblasts by TGF-α1. Normal human lung fibroblasts (IMR-90) were stimulated ±TGF-â1 (2 ng/ml) Gleevec at the doses indicated for 24 h. Cell lysates were then subjected to SDS-PAGE and immunoblotted for specific antibodies to the proteins shown.

FIG. 10 shows the effects of the PKB/Akt inhibitor, NL-71-101, on bleomycin-induced pulmonary fibrosis. Bleomycin-injured mice were treated with/without NL-71-101 at a dose of 10 mg/kg by daily IP injections starting on day 8 following injury. Tissue sections were stained (H&E, mag 100×) 21 days following bleomycin and 14 days after treatment with NL-71-101.

### DEFINITIONS

To facilitate an understanding of the present invention, a number of terms and phrases are defined below.

As used herein, the term “protein kinase” refers to a member of an enzyme superfamily which functions to phosphorylate one or more protein as described above.

As used herein, the term “activated PKB/Akt” or “activated protein kinase” or similar term refers to a protein kinase which is phosphorylated.

As used herein, the term “inhibitor” is interchangeably used to denote “antagonist;” these terms define compositions that have the capability of decreasing certain enzyme activity or competing with the activity or function of a substrate of said enzyme.

As used herein, the term “fibrous disease,” “fibrotic disorder,” “fibrotic disease,” and “fibrotic disorder” broadly refer an excessive production of extracellular matrix and profibrotic proteins such as fibroblasts and myofibroblasts. Examples of fibrous diseases include, but are not limited to, idiopathic pulmonary fibrosis, pulmonary fibrosis, acute respiratory distress syndrome, liver disease, liver cirrhosis and hepatitis C, renal interstitial fibrosis, cystic fibrosis, pancreatic fibrosis, keloid, secondary fibrosis in the gastrointestinal tract, hypertrophic burn scars, myocardial fibrosis, Alzheimer's disease, retinal detachment inflammation and/or fibrosis resulting after surgery, graft versus host and host versus graft rejections, systemic sclerosis, systemic fibrosing disease, complicated silicosis, renal fibrosing disease, pulmonary diseases, renal diseases, pathologic skin scarring as colloid and hypertrophic scar, cirrhosis of liver and gallbladder, pulmonary and bone-marrow fibrosis, scleroderma, sarcoidosis, and keloids.

As used herein, the term “pyrazolopyrimidine” broadly refers to a Src family tyrosine kinase inhibitor. In some embodiments the present invention contemplates, but is not limited to, the pyrazolopyrimidine compounds described in U.S. Pat. Nos. 6,833,371, 6,730,680, 6,660,744, 6,664,261, 6,194,410, 6,051,578; U.S. Patent Application Nos. 20040209878A1, 20040191824A1, 20040127508A1, 20040127483A1, 2004011644A1, 20040106624A1, 20040102452A1, 20040102451A1, 20040006083A1; and International Patent Application Nos: WO05030773A1, WO04113344A1, WO04106341A1, WO04099211 A1, WO04099210A1, WO04087707A1, WO04064721A3, WO04083211A1, WO04022062A1, WO04009602A1, WO04018474A1, WO03095455A3, WO03080617A1, WO03076441A1 are utilized; each herein incorporated by reference in their entireties. In some aspects, the structure of a pyrazolopyrimidine is presented below.

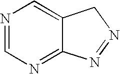

The term “derivative” of a compound, as used herein, refers to a chemically modified compound wherein the chemical modification takes place either at a functional group of the compound or on a non functional group. A non limiting example of a pyrazolopyrimidine derivative is AG1879.

The term “AG1879” or “PP2” refers to a pyrazolopyramidine compound that potently inhibits the Src family kinases and integrin-dependent FAK activation. The chemical formula for AG1979 is 4-amino-5-(4-chlorophenyl)-7-(t-butyl)pyrazolo[3,4-d]pyrimidine, and is presented below.

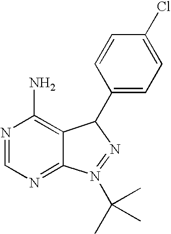

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of pyrazolopyrimidine compound(s), and optionally one or more other agents) for a condition characterized by the activation of PKB/Akt.

The term “diagnosed,” as used herein, refers to the to recognition of a disease by its signs and symptoms (e.g., resistance to conventional therapies), or genetic analysis, pathological analysis, histological analysis, and the like.

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

In preferred embodiments, the “target cells” or “target tissues” of the compositions and methods of the present invention include, refer to, but are not limited to, fibrotic cells, lung cells, fibrotic tissue, or lung tissue. In some embodiments, target cells are continuously cultured cells or uncultured cells obtained from patient biopsies.

In one specific embodiment, the target cells exhibit pathological growth or proliferation. As used herein, the term “pathologically proliferating or growing cells” refers to a localized population of proliferating cells in an animal that is not governed by the usual limitations of normal growth.

As used herein, the term “effective amount” refers to the amount of a compound (e.g., AG1879) sufficient to effect beneficial or desired results. An effective amount can be administered in one or more administrations, applications or dosages and is not limited intended to be limited to a particular formulation or administration route.

As used herein, the term “co-administration” refers to the administration of at least two agent(s) (e.g., pyrazolopyrimidines) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “solid phase supports” or “solid supports,” are used in their broadest sense to refer to a number of supports that are available and known to those of ordinary skill in the art. Solid phase supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid phase support may be selected on the basis of desired end use and suitability for various protocols. For example, for peptide synthesis, solid phase supports may refer to resins such as polystyrene (e.g., PAM-resin obtained from Bachem, Inc., Peninsula Laboratories, etc.), POLYHIPE) resin (obtained from Aminotech, Canada), polyamide resin (obtained from Peninsula Laboratories), polystyrene resin grafted with polyethylene glycol (TENTAGEL, Rapp Polymere, Tubingen, Germany) or polydimethylacrylamide resin (obtained from Milligen/Biosearch, Calif.).

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms which are gram negative or gram positive. “Gram negative” and “gram positive” refer to staining patterns with the Gram-staining process which is well known in the art. (See e.g., Finegold and Martin, Diagnostic Microbiology, 6th Ed., CV Mosby St. Louis, pp. 13-15 [1982]). “Gram positive bacteria” are bacteria which retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

As used herein, the term “virus” refers to minute infectious agents, which with certain exceptions, are not observable by light microscopy, lack independent metabolism, and are able to replicate only within a living host cell. The individual particles (i.e., virions) typically consist of nucleic acid and a protein shell or coat; some virions also have a lipid containing membrane. The term “virus” encompasses all types of viruses, including animal, plant, phage, and other viruses.

The term “sample” as used herein is used in its broadest sense. A sample suspected of indicating a condition characterized by the activation of PKB/Akt may comprise a cell, tissue, or fluids, genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., the level of PKB/Akt activation). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In preferred embodiments, “test compounds” are agents that modulate PKB/Akt activation in cells.

### DETAILED DESCRIPTION OF THE INVENTION

The present invention provides novel chemical compounds, methods for their discovery, and their therapeutic use. In particular, the present invention provides pyrazolpyrimidine compounds, pyrazolopyrimidine derivatives and related compounds and methods of using pyrazolopyrimidine compounds, pyrazolopyrimidine derivatives, and related compounds as therapeutic agents to treat a number of conditions associated with fibrotic disorders.

Exemplary compositions and methods of the present invention are described in more detail in the following sections: I. Modulators of PKB/Akt; II. Exemplary Compounds; III. Pharmaceutical compositions, formulations, and exemplary administration routes and dosing considerations; and IV. Drug screens.

The practice of the present invention employs, unless otherwise indicated, conventional techniques of organic chemistry, pharmacology, molecular biology (including recombinant techniques), cell biology, biochemistry, and immunology, which are within the skill of the art.

### I. Modulators PKB/Akt

The emergence of altered cellular phenotypes is a characteristic of many chronic human disease processes such as cancer, airway remodeling (e.g., asthma), vascular remodeling (e.g., atherosclerosis/pulmonary hypertension) and parenchymal tissue fibrosis. Phenotypic alterations of key “effector” cells are critical to pathogenesis and progression of these diseases. The pathogenesis of fibrotic disorders is linked to “activated” fibroblast phenotypes such as myofibroblasts in diverse tissues and organ systems, including the kidney, liver and lung (see, e.g., Border, W. A., J Clin Invest 90:1-7 (1992); Border, W. A., et al., N Engl J Med 331:1286-1292 (1994); Iwano, M., et al., J Clin Invest 110:341-350 (2002); Pan, D., et al., J Clin Invest 110:1349-1358 (2002); each herein incorporated by reference in their entireties).

Fibroblast recruitment and activation represent a normal response to tissue injury and resolution of injury and healing without fibrosis is associated with apoptosis of myofibroblasts (see, e.g., Desmouliere, A., et al., Am J Pathol 146:56-66 (1995); herein incorporated by reference in its entirety). Persistence of the fibroblasts/myofibroblasts in injured tissues is a consistent finding in diseases characterized by progressive fibrosis.

In experiments conducted during the development of the present invention, constitutive activation of the protein kinases, FAK and PKB/Akt, was detected in fibroblasts isolated and cultured ex vivo from the lungs of patients with IPF. FAK and PKB/Akt pathways are critically involved in pro-survival/anti-apoptotic signaling (see, e.g., Cantley, L. C. Science 296:1655-1657 (2002); Giancotti, F. G., et al., Science 285:1028-1032 (1999); Brazil, D. P., et al., Cell 111:293-303 (2002); each herein incorporated by reference in their entireties). The present invention is not limited to a particular mechanism. Indeed, an understanding of the mechanism is not necessary to practice the present invention. Nonetheless, based upon experiments conducted during the course of the present invention, the FAK and PKB/Akt pathways are involved in the persistence/activation of mesenchymal cells during fibrogenic processes.

In experiments conducted during the development of the present invention, activation of PKB/Akt and FAK was detected after exposure to TGF-β1. TGF-β1 is a potent fibrogenic cytokine (see, e.g., Border, W. A., et al., J Clin Invest 90:1-7 (1992); Border, W. A., et al., J Clin Invest 96:655-656 (1995); Border, W. A., et al., N Engl J Med 331:1286-1292 (1994); each herein incorporated by reference in their entireties).

The present invention is not limited to a particular mechanism. Indeed, an understanding of the mechanism is not necessary to practice the present invention. Nonetheless, based upon experiments conducted during the course of the present invention, the mechanism(s) by which FAK is activated by TGF-β1 is, in part, related to the ability of this cytokine to induce the expression of integrin receptors and ECM production, in particular, fibronectin (see, e.g., Thannickal, V. J., et al., J Biol Chem 278:12384-12389 (2003); each herein incorporated by reference in its entirety). In addition, PI3K-Akt activation is dependent on the early activation of Smad-independent p38 MAPK activation by TGF-β1 (see, e.g., Thannickal, V. J. et al, J. Biol. Chem., 279:1359-1367 (2004); herein incorporated by reference in its entirety).

The murine model of bleomycin-lung injury is a well-established model of pulmonary fibrosis (see, e.g., Nakao, A., et al., J Clin Invest 104:5-11 (1999); Eitzman, D. T., et al., J Clin Invest 97:232-237 (1996); Huang, M., et al., J Clin Invest 109:931-937 (2002); each herein incorporated by reference in their entireties). Key pathogenetic mechanisms in human disease such as the expression/activation of TGF-β1 and induction of myofibroblasts are important features in the murine model (see, e.g., Zhang, K., et al., Am J Pathol 147:352-361 (1995); herein incorporated by reference in its entirety). Myofibroblast presence/activation in association with TGF-β1 expression is transient in the murine model and decreases with concomitant reduction in fibrosis (see, e.g., Zhang, K., et al., Am J Pathol 147:352-361 (1995); herein incorporated by reference in its entirety). Persistence of myofibroblasts and TGF-β1 over-expression are characteristic of progressive fibrotic disease in humans (see, e.g., Border, W. A., et al., J Clin Invest 90:1-7 (1992); herein incorporated by reference in its entirety).

In preferred embodiments of the present invention, compositions comprising pyrazolopyrimidine compounds are used to modulate fibrosis. The present invention is not limited to a particular type of pyrazolopyrimidine. In preferred embodiments, AG1879 is used. In further embodiments, AG1879 modulates fibrosis rather than inflammation. In preferred embodiments, AG1879 inhibits PKB/Akt activation. In further embodiments, administration of AG1879 decreases PKB/Akt phosphorylation in fibroblasts cultured ex vivo from mice treated with AG1879.

In some embodiments, the NL-71-101 (Cal Biochem) compound and NL-71-101 derivative compounds find use in the present invention. NL-71-101 is provided below:

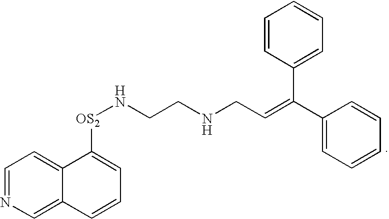

NL-71-101 is a potent, ATP-competitive, and selective inhibitor of protein kinase B/Akt. Additionally, NL-71-101 has been shown to induce apoptosis in OVCAR-3 ovarian cancer cells in which PKB is strongly amplified. NL-71-101 is also shown to inhibit phosphorylation of the PKB/Akt substrate, glycogen synthase kinase-3 (GSK3) in intact cells (see, e.g., Reuveni, H. et al., Biochemistry 41, 10304 (2002); herein incorporated by reference in its entirety). In preferred embodiments, the NL-71-101 compound is used in the treatment of fibrosis (e.g., bleomycin-induced pulmonary fibrosis).

### II. Exemplary Compounds

The present invention provides pyrazolopyrimidine compounds. The present invention is not limited to particular pyrazolopyrimidine compound. In some embodiments, the pyrazolopyrimidine compounds described in U.S. Pat. Nos. 6,833,371, 6,730,680, 6,660,744, 6,664,261, 6,194,410, 6,051,578; U.S. Patent Application Nos. 20040209878A1, 20040191824A1, 20040127508A1, 20040127483A1, 2004011644A1, 20040106624A1, 20040102452A1, 20040102451A1, 20040006083A1; and International Patent Application Nos: WO05030773A1, WO04113344A1, WO04106341A1, WO04099211A1, WO04099210A1, WO04087707A1, WO04064721A3, WO04083211A1, WO04022062A1, WO04009602A1, WO04018474A1, WO03095455A3, WO03080617A1, WO03076441A1 are utilized; each herein incorporated by reference in their entireties. In preferred embodiments, the present invention provides combinations, derivatives, and pharmaceutical combinations of pyrazolopyrimidine compounds. In preferred embodiments, the present invention utilizes the pyrazolopyrimidine compound AG1879 (4-Amino-5-(4-chlorophenyl)-7-(t-butyl)pyrazolo[3,4-d]pyrimidine).

### III. Pharmaceutical Compositions, Formulations, and Exemplary Administration Routes and Dosing Considerations

Exemplary embodiments of various contemplated medicaments and pharmaceutical compositions are provided below.

A. Preparing Medicaments

The compounds of the present invention are useful in the preparation of medicaments to treat a variety of conditions associated with a fibrous disease (e.g., idiopathic pulmonary fibrosis). The methods and techniques for preparing medicaments of a compound are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts (see, e.g., U.S. Pat. Nos. 6,660,744, 6,664,261, 6,194,410, 6,051,578; U.S. Patent Application No. 20040006083A1; and International Patent Application Nos. WO04022062A1, WO04009602A1, WO04018474A1, WO03095455A3, WO03080617A1, WO03076441A1 are utilized; each herein incorporated by reference in their entireties).

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent (e.g., pyrazolopyrimidine derivative), as defined above, together with a solid support or alternatively, together with one or more pharmaceutically acceptable carriers and optionally other therapeutic agents. Each carrier should be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In preferred embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In preferred embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in an known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

C. Exemplary Administration Routes and Dosing Considerations

Various delivery systems are known and can be used to administer therapeutic agents (e.g., AG1879 derivatives) of the present invention, e.g., encapsulation in liposomes, microparticles, microcapsules, receptor-mediated endocytosis, and the like. Methods of delivery include, but are not limited to, intra-arterial, intramuscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals susceptible to or at risk of developing pathological growth of target cells and condition correlated with this. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is a fibrotic disorder (e.g., idiopathic pulmonary fibrosis), the additional agent can be an anti-fibrotic agent (e.g., colchicines), an antiviral cytokine (e.g., interferon-gamma-1b), an immunosuppressive agent (e.g., azathioprine), and/or a glucocorticoid (e.g., prednisone). In addition, combinations of such agents with pyrazolopyrimidine agents are contemplated. The determination of appropriate type and dosage of treatment is also within the skill in the art or can be determined with relative ease.

### IV. Drug Screens

In preferred embodiments of the present invention, the compounds of the present invention, and other potentially useful compounds, are screened for their ability to inhibit the activation of PKB/Akt and FAK. A number of suitable screens for measuring the activation or inhibition of protein kinases are known in the art. In some embodiments, protein kinase activation screens are conducted in in vitro systems. In other embodiments, these screens are conducted in in vivo or ex vivo systems. The present invention is not limited to a particular type of screening assay. In preferred embodiments, PKB activity was measured through phosphorylation assays of diseased (e.g., fibrotic) tissues (see, e.g., Example XIII). In preferred embodiments, the PKB activity assays described in International Patent Application WO03010281 are utilized (e.g., PKB in vitro kinase activity assays, transfer ELISA assays for measuring PKB activity). In further preferred embodiments, the PKB inhibition assays described International Patent Application WO03010281 are utilized (e.g., Annexin-V-apoptosis assays, ELISA assay for detection of ssDNA-apoptosis assays, cell viability assays, phosphorylation inhibition assays).

### EXAMPLES

The following examples are provided to demonstrate and further illustrate certain preferred embodiments of the present invention and are not to be construed as limiting the scope thereof.

### Example I

### Isolation and Culture of Human Lung Fibroblasts

Research protocols involving human subjects received prior approval by the Institutional Review Board at the University of Michigan. Fibroblasts were isolated by explant cultures of surgical lung biopsies demonstrating histopathological findings of usual interstitial pneumonia (UIP) from patients with a clinical diagnosis of IPF as defined by the American Thoracic Society/European Respiratory Society (see, e.g., American Thoracic Society, Am J Respir Crit. Care Med 161:646-664 (2000); herein incorporated by reference in its entirety). Fibroblasts were also isolated from normal-appearing lung tissue sections surgically removed from patients undergoing evaluation for suspected lung cancer. Cells were cultured in medium consisting of Dulbecco's modified Eagle's medium (DMEM; GIBCO, Grand Island, N.Y.) supplemented with 10% fetal bovine serum (FBS; Sigma, St. Louis, Mo.), 100 U/ml penicillin/streptomycin (Sigma, St. Louis Mo.), and fungizone (GIBCO, Grand Island, N.Y.); medium was changed every two days. Passage 3-5 fibroblasts were plated on 60 mm cell culture dishes at a density of 5×10⁵ cells/dish and incubated at 37° C. in 5% CO₂-95% air. When cells reached 80% confluence, they were growth-arrested for 48 hours in DMEM with 0.01% FBS prior to treatment with/without TGF-β1.

### Example II

### Isolation and Culture of Murine Lung Fibroblasts

Mice were euthanized by CO₂ asphyxiation and perfused via the heart with 5 ml of normal saline. Whole lungs were sterilely removed and cut into small 2- to 3-mm slices and allowed to adhere on tissue culture plastic. Lung tissue explants were maintained in medium consisting of Dulbecco's modified Eagle's medium (Life Technologies, Inc.) supplemented with 10% fetal bovine serum (Sigma), 100 U/ml penicillin/streptomycin (Sigma), and fungizone (Life Technologies, Inc.). Fibroblasts were purified by repeat trypsinization and passaging to achieve a homogenous population of spindle cells that uniformly expressed the collagen cross-linking enzyme, prolyl 4-hydroxylase (see, e.g., Konttinen, et al., (1989) J. Rheumatol. 16:339-345; herein incorporated by reference in its entirety). Cell lysates were obtained for Western blot analyses at passage 2 to 3 and confluency of 90 to 100%.

### Example III

### Reagents and Drugs

Porcine-derived TGF-β1 was obtained from R&D Systems, Minneapolis, Minn. All other cell culture reagents were from Sigma, St Louis, Mo. AG1879 {PP2: 4-Amino-5-(4-chlorophenyl)-7-(t-butyl)pyrazolo[3,4-d]pyrimidine} and its inactive analog, AG/inactive {PP3: 4-Amino-7-phenylpyrazol[3,4-d]pyramidine} were purchased from Calbiochem, La Jolla, Calif. Drugs were initially solubilized in DMSO to make a 30 mM “stock” concentration. Cell culture experiments were performed at final concentrations ranging from 1-10 μM by diluting in cell culture medium. For animal experiments, the same stock concentration of AG1879 was further diluted in normal saline to make a final concentration of 0.7 mg/ml. Daily intraperitoneal injections of mice were with 0.25 ml of this final mixture (or, 0.175 mg/mouse) using a 26-gauge sterile intradermal needle. This dose was based on the observed efficacy of the drug at concentrations ≧3 μM in cell culture systems. The calculated dose (0.175 mg/injection) would achieve 3.0 μM concentration of the drug in a volume (ml) of distribution equivalent to the weight (mg) of the mice, assuming 100% bioavailability of the drug and average weight of mice of 19 gm. Dose of AG/inactive (control) drug was calculated to achieve the same concentration as active AG1879. In early pilot experiments, bleomycin-injured mice tolerated this dose without evidence of toxicity and, in fact, AG1879-treated mice appeared to be more active and gained more weight than control drug-treated and untreated animals.

### Example IV

### Western Immunoblotting and Antibodies

Cultured cells were washed in cold PBS and lysed in ice-cold RIPA lysis buffer (1% NP-40, 1% sodium deoxycholate, 0.1% SDS, 0.15 M NaCl, 0.01 M NaH₂PO₄, 2 mM EDTA, 0.5 mM NaF) containing 2 mM sodium orthovanadate and 1:100 dilution of protease inhibitor cocktail III (Calbiochem, La Jolla, Calif.). Cell lysates were then subjected to SDS-PAGE and Western blot analyses performed as previously described (see, e.g., Thannickal, V. J., et al., J Biol Chem 273:23611-23615 (1998); herein incorporated by reference in its entirety). Rabbit polyclonal antibodies to phospho-Akt (Ser473), total Akt, and total ERK (p44/42) were from Cell Signaling Technology. Mouse monoclonal antibody to phospho-p44/42 (Thr202/Tyr204) was from Cell Signaling Technology. Phosphorylation-specific antibody to tyrosine-397 FAK was from Biosource International, Carmillo, Calif. Antibody to total FAK was from Santa Cruz Biotechnology, Santa Cruz, Calif. Secondary horseradish peroxidase (HRP)-conjugated anti-goat, anti-mouse, and anti-rabbit antibodies were obtained from Pierce, Rockford, Ill.

### Example V

### Mice and Bleomycin Injury Model

B6129F2/J mice were purchased from Jackson Laboratories, Bar Harbor, Me., and housed under specific pathogen free conditions in enclosed filter top cages. Clean food and water was given ad libitum. The mice were handled and maintained using microisolator techniques with daily veterinarian monitoring. The University of Michigan Committee on the Use and Care of Animals (UCUCA) approved these experiments. Intra-tracheal bleomycin was administered to mice as previously described (see, e.g., Kuwano, K., et al., J Clin Invest 104:13-19 (1999); Hattori, N., et al., J Clin Invest 106:1341-1350 (2000); each herein incorporated by reference in their entireties) with minor modifications. A single 30 μl aliquot containing 0.025 U of bleomycin (Sigma, St. Louis, Mo.) diluted in normal saline was intra-tracheally injected using a Tridak stepper (Brookfield, Conn.) and a 30-gauge needle.

### Example VI

### Sircol Assay for Collagen

Mice were euthanized by CO₂ asphyxiation and perfused via the heart with 5 ml of normal saline. Whole lungs were removed, taking care to avoid the large conducting airways and homogenized in 1 ml of 0.5% Triton X-100. After centrifugation, 100 μl of supernatant was mixed with 1 ml of Sircol collagen assay dye reagent (30 minutes at room temperature). Following centrifugation, the pellet was resuspended in 1 ml of alkali reagent, vortexed to release the dye into solution, and 100 μl transferred to a microplate and absorbance measured at 540 nm. Values for experimental samples were calculated based on a standard curve of known concentrations of purified rat tail collagen.

### Example VII

### Lung Histology

Animals were euthanized, and perfused via the right ventricle with 3 ml normal saline. Lungs were inflated with 1 ml 10% neutral buffered formalin, removed, and fixed overnight in formalin before being dehydrated in 70% ethanol. Lungs were processed using standard procedures and embedded in paraffin. 3-5 micron sections were cut, mounted on slides, and stained with hematoxylin and eosin (H & E) or Masson's trichrome blue for collagen.

### Example VIII

### Immunohistochemical Staining

Sections from paraffin-embedded tissues for all of the treatment groups were processed for immunohistochemical localization of α-smooth muscle actin (α-SMA) to identify myofibroblasts. The slides were also immunostained with the same phospho-specific antibodies to the phosphorylated (activated) isoforms of PKB/Akt and FAK. The tissue sections were dewaxed and were exposed to heat-induced antigen retrieval treatment using a Tendercook pressure cooker and heat antigen unmasking solution (Biogenex, San Ramon, Calif.) in the microwave for 13.5 minutes. Subsequently, the slides were stained using a sensitive avidin-streptavidin-peroxidase in an automated cell staining system (GenoMx model i6000; Biogenex, San Ramon, Calif.). The sections were then counterstained with hematoxylin and mounted. Photomicrographs were taken at ×200 magnification.

### Example IX

### Collagenase Digestions of Whole Lung

Collagenase digestions can be used to analyze both resident and recruited populations of lung cells found both in the alveolar space and interstitium. This procedure has been optimized to purify lung leukocytes (see, e.g., Huffnagle, G. B., et al., J Immunol 155:4790-4797 (1995); herein incorporated by reference in its entirety). Lungs were excised, minced, and enzymatically digested for 30 minutes using 15 ml/lung of digestion buffer (RPMI, 5% FCS, antibiotics, 1 mg/ml collagenase (Boehringer Mannheim Corp., Chicago, Ill.) and 30 μg/ml DNAse (Sigma, St. Louis, Mo.). The cell suspension and undigested fragments were further dispersed by repeated passage through the bore of a 10-ml syringe without a needle. The total cell suspension was pelleted, and any contaminating erythrocytes were eliminated by lysis in ice-cold NH₄Cl buffer (0.829% NH₄Cl, 0.1% KHCO₃, and 0.0372% Na₂ EDTA, pH 7.4). The pellet was resuspended in 5 ml of complete medium (RPMI, 5% FBS, 1% penicillin/streptomycin) and dispersed by 20 passages through a 5 ml syringe. The dispersed cells were filtered through a Nytex filter (Tetko, Inc., Kansas City, Mo.) to remove clumps. The total volume was brought up to 10 ml with complete media. An equal volume of 40% Percoll (Sigma, St. Louis, Mo.) was added and the cells were centrifuged at 3000 rpm for 30 minutes (room temperature) without a brake. The cell pellets were resuspended in complete media, and leukocytes were counted on a hemocytometer in the presence of trypan blue. Cells were greater than 90% viable by trypan blue exclusion. Cytospins of recovered cells were prepared for differential staining as described below.

### Example X

### Differential Staining

Cytospins of collagenase digests were made by centrifugation of 50,000 cells on microscope slides using a Shandon Cytospin 3 (Astmoore, England). The slides were allowed to air dry and were stained using a modified Wright-Giemsa (WG) stain. For WG staining, the slides were fixed/prestained for 2 minutes with a one-step methanol-based WG stain (Harleco; EM Diagnostics, Gibbstown, N.J.), followed by steps 2 and 3 of the Diff-Quick whole blood stain (Diff-Quick; Baxter Scientific, Miami, Fla.). This modification of the Diff-Quick stain procedure improves the resolution of eosinophils from neutrophils in the mouse. A total of 300 cells were counted from randomly chosen from high-power microscope fields for each sample. The differential percentage of each cell type was multiplied by the total leukocyte count to derive an absolute number of monocyte/macrophages, neutrophils and eosinophils per sample.

### Example XI

### Densitometric Analyses

Digital images of Western blots were scanned and band intensities analyzed using NIH Image public domain software (http://rsb.info.nih.gov/nih-image). Ratios of phospho-specific to total protein were calculated for each set of samples.

### Example XII

### Statistical Analyses

Statistical significance was analyzed using the InStat 2.01 program (Graphpad Software). Student's t-tests were run to determine p values when comparing two groups. When comparing 3 or more groups, ANOVA analysis was performed with a post-hoc Bonferroni test to determine which groups showed significant differences; p<0.05 was considered significant.

### Example XIII

### PKB/Akt and FAK are Constitutively Activated and Regulated by TGF-β1 in Fibroblasts Isolated from IPF Patients

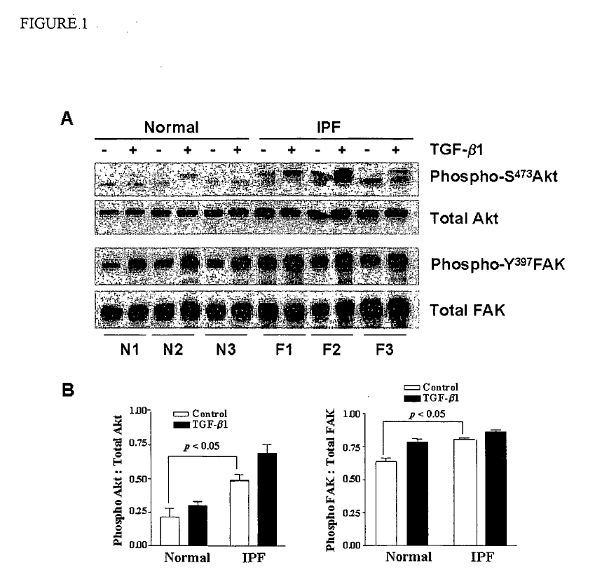

Cells in “diseased” tissues are phenotypically altered by dynamic changes in tissue microenvironments, thereby, contributing to disease pathogenesis. Fibroblasts were isolated from diseased portions of lungs of IPF patients (IPF-fibroblasts) and unaffected, normal-appearing lungs of patients undergoing evaluation for lung cancer (normal-fibroblasts). Cells were cultured ex vivo for 3-5 passages. Subconfluent cultures were then analyzed for the expression of activated (phosphorylated) PKB/Akt and FAK, protein kinases that mediate pro-survival and pro-fibrotic fibroblast phenotypes (see, e.g., Thannickal, V J., et al., J Biol Chem 278:12384-12389 (2003); Kim, G, et al., Arthritis Rheum 46:1504-1511 (2002); each herein incorporated by reference in their entireties). FIG. 1 demonstrates that the baseline levels (in the absence of TGF-β1) of PKB/Akt and FAK phosphorylation are increased in IPF-fibroblasts when compared to normal-fibroblasts. This difference is appreciated with culture of all cells under identical ex vivo conditions, suggesting stable changes in the “signaling program” of these cells. Differential activation of these pathways in IPF-fibroblasts appears to be greater in the case of PKB/Akt than for FAK (>2-fold increase in constitutive phosphorylation in IPF vs. normal; FIG. 1B). Exogenous TGF-β1 (2 ng/ml×16 h) enhances phosphorylation of PKB/Akt and FAK in all groups except for the induction of PKB/Akt phosphorylation in normal-fibroblasts which showed an upward trend that did not reach statistical significance (FIG. 1B).

### Example XIV

### Constitutive- and TGF-β1-Induced Activation of PKB/Akt and FAK are Inhibited by AG1879

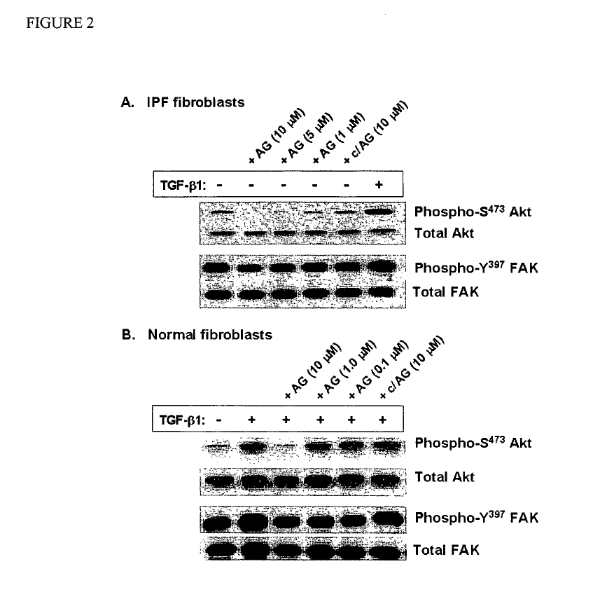

PKIs exert their effects with varying degrees of specificity (see, e.g., Davies, S. P., et al., Biochem J 351:95-105 (2000); Bain, J., et al., Biochem J 371:199-204 (2003); each herein incorporated by reference in their entireties). AG1879 is a pyrazolopyramidine compound that potently inhibits the Src family kinases and integrin-dependent FAK activation (see, e.g., Thannickal, V. J., et al., J Biol Chem 278:12384-12389 (2003); Salazar, E. P., J Biol Chem 276:17788-17795 (2001); each herein incorporated by reference in their entireties), and less potently other protein kinases (see, e.g., Bain, J., et al., Biochem J 371:199-204 (2003); each herein incorporated by reference in their entireties). The effect of AG1879 on the constitutive phosphorylation of PKB/Akt and FAK in IPF-fibroblasts was examined and on the TGF-β1-induced activation of these protein kinases in normal-fibroblasts. AG1879 dose-dependently inhibited the constitutive PKB/Akt phosphorylation in IPF fibroblasts with almost complete inhibition at 10 μM AG1879, added for 16 h prior to cell lysis (FIG. 2A, top panels). The inactive analog of AG1879 (control, c/AG) had no effect, whereas TGF-β1 was able to upregulate constitutive levels of PKB/Akt phosphorylation. A dose-dependent inhibition of constitutive FAK phosphorylation was also noted, but this was less marked than the inhibitory effects of AG1879 on PKB/Akt phosphorylation. To determine the effects of AG1879 on TGF-β1-induced activation of PKB/Akt and FAK in normal-fibroblasts, AG1879 was co-treated at the doses noted (log-scale) and examined phosphorylation at 16 h. The TGF-β1-induced phosphorylation of both PKB/Akt and FAK were inhibited to baseline levels with 10 μM AG1879, whereas control (inactive compound, c/AG) had no effect (FIG. 2B).

### Example XV

### FAK and PKB/Akt Protein Kinases are Activated in Fibrotic Foci of Bleomycin-Injured Murine Lung and are Inhibited by Systemic Administration of AG1879

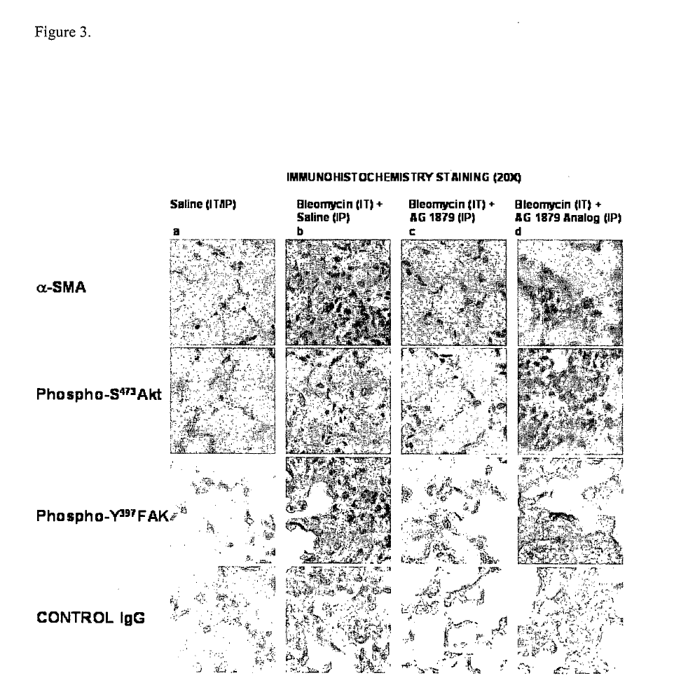

The protein kinases, PKB/Akt and FAK, were shown to be upregulated in fibrotic areas in bleomycin-injured lung. Additionally, administration of the anti-fibrotic PKI, AG1879, attenuated activation of PKB/Akt and FAK in vivo (see FIG. 3).

Intratracheal instillation of bleomycin in mice induces an acute lung injury followed by well-defined inflammatory and fibrotic phases (see, e.g., Izbicki et al., (2002) Int. J. Exp. Pathol. 83:111-119; herein incorporated by reference in its entirety). This animal model of pulmonary fibrosis does not replicate all of the features of human IPF (see, e.g., Borzone, et al., (2001) Am. J. Respir. Crit. Care Med. 163:243-252; herein incorporated by reference in its entirety), but is useful in studying certain pathophysiological mechanisms. Importantly, fibrosis in this model is associated with enhanced TGF-β1 expression/activation and the emergence of myofibroblasts (see, e.g., Zhang, et al., (1994) Am. J. Pathol. 138:1257-1265; herein incorporated by reference in its entirety), typical of human fibrotic disorders (see, e.g., Thannickal, et al., (2004) 55:395-417; Tomasek, et al., (2002) Nat. Rev. Mol. Cell. Biol. 3:349-363; each herein incorporated by reference in their entireties).

Daily intraperitoneal injections of AG1879 (175 μg/mouse; ˜10 mg/kg) or its inactive analog (AG 1879/inactive; same dose) were administered starting a week after bleomycin injury. This time point was selected based on relative decline in inflammation and activation of fibrogenic responses including myofibroblast emergence and persistence. After 7 days of AG1879 treatment, lungs were harvested and tissue sections examined for the activational state of PKB/Akt and FAK by IHC staining with phospho-specific antibodies against the activated forms of these protein kinases; representative sections were also immunostained for α-SMA. Focal areas of dense cellularity and fibrosis contained cells that express α-SMA, a marker of myofibroblasts (FIG. 3). Cells in these areas of active tissue fibrosis strongly expressed phosphorylated (activated) PKB/Akt and FAK (FIG. 3). Systemic administration of AG1879 to injured mice attenuated the activation of these protein kinases in vivo in association with markedly reduced fibrotic responses (FIG. 3).

### Example XVI

### Modulation of Lung Fibroblast Signaling/Phenotype in Bleomycin Injured Mice with AG1879

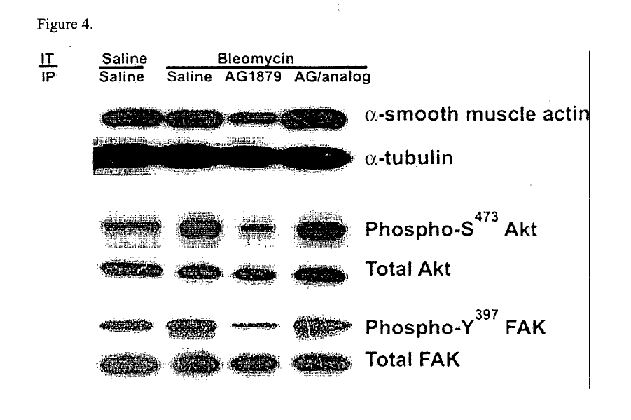

To determine the result of systemic administration of AG1879 in stable modulation of signaling/phenotype of fibroblasts and myofibroblasts in vivo, fibroblasts isolated from lungs of bleomycin injured mice were analyzed. Fibroblasts were isolated on day 15 after lung injury by explant cultures and adherence purification. Studies were performed on relatively pure (˜99% by staining for the collagen crosslinking-enzyme, prolyl-4-hydroxylase (see, e.g., Konttinen, et al., (1989) J. Rheumatol. 16:339-345; herein incorporated by reference in its entirety) and by cell morphology) fibroblast populations at passage 2. AG1879 was administered (intraperitoneally) for 7 days, starting a week after initial bleomycin injury. Constitutive expression of activated/phosphorylated FAK and PKB/Akt as well as expression of α-SMA were assessed by Western immunoblots using whole cell (RIPA) lysates. Fibroblasts isolated from bleomycin-injured mice showed elevated levels of α-SMA, indicating enhanced myofibroblast differentiation. This effect was partially inhibited by AG1879 treatment (see FIG. 4). Bleomycin injury also induced stable up-regulation of PKB/Akt and FAK phosphorylation in fibroblasts isolated at the time (day 15) of active in vivo fibrogenesis. This effect was attenuated in lung fibroblasts of mice treated with AG1879 (FIG. 4).

### Example XVII

### Systemic Administration of AG1879 Protects Against Bleomycin-Induced Lung fibrosis in Mice

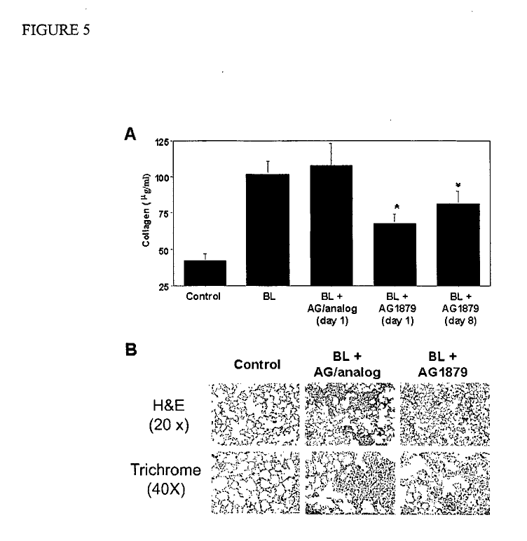

PKIs have therapeutic benefit in diseases characterized by abnormal cell phenotypes associated with dysregulated protein kinase signaling (see, e.g., Cohen, P. Nat Rev Drug Discov 1:309-315 (2002); herein incorporated by reference in its entirety). To determine if systemic administration of AG1879 attenuates fibrotic reactions to lung injury, the effects of this PKI in a murine model of bleomycin-induced pulmonary fibrosis was examined (see, e.g., Kuwano, K., et al., J Clin Invest 104:13-19 (1999); Hattori, N., et al., J Clin Invest 106:1341-1350 (2000); each herein incorporated by reference in their entireties). Daily intra-peritoneal injections of AG1879 (175 μg/mice; ˜10 mg/kg) or its inactive analog (AG 1879/inactive; same dose) were administered starting either on the day of injury (day 1) or a week later (day 8). Mice receiving AG1879 (BL-AG1879) developed less fibrosis than mice receiving control drug (BL-AG1879) as assessed by accumulation of total lung collagen at day 14 (FIG. 5A). Significant protection was observed both when the drug was given at early (day 1) and late (day 8) following BL-injury (FIG. 5A). Lung histology (hematoxylin and eosin staining; FIG. 5B, top panels) showed significantly less fibrotic tissue reactions in BL-AG1879 than mice receiving control drug. Masson's trichrome staining for collagen also demonstrated marked attenuation in accumulated collagen in mice receiving active vs. inactive drug (FIG. 5B, bottom panels). The present invention is not limited to a particular mechanism. Indeed, an understanding of the mechanism is not necessary to practice the present invention. Nonetheless, these results demonstrate that systemic administration of the PKI, AG1879, protects against the development of fibrotic lung injury in vivo. Importantly, this protection is observed both when the drug is administered early and in a delayed manner relative to the onset of injury.

### Example XVIII

### Protection Against Fibrotic Lung Injury by AG1879 is not Associated with Alterations in the Inflammatory Response

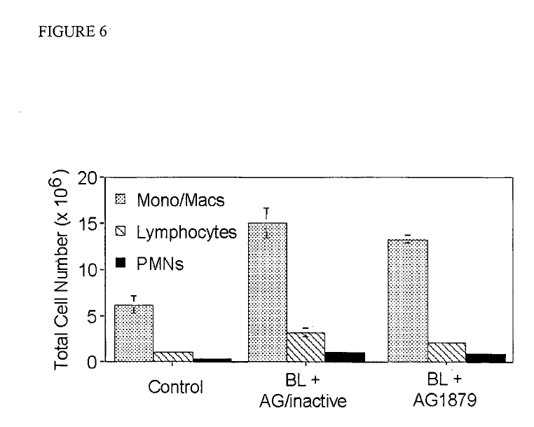

The murine model of bleomycin injury is associated with an early inflammatory response that persists up to 7 days. To determine if the observed protection against fibrosis was related to alterations in the magnitude of the inflammatory response, the number of inflammatory cells on day 7 was assessed by collagenase digest. Bleomycin-injured mice receiving inactive drug (BL+AG/inactive) developed typical increases in monocytes/macrophages, lymphocytes and lesser increases in polymorphonuclear cells (PMNs) compared to saline (no bleomycin) controls. Administration of the active PKI, AG1879 (BL+AG1879), starting on the initial day of bleomycin injury did not significantly alter the number of recruited/resident inflammatory cells of any of the subpopulations examined (FIG. 6).

### Example XIX

### In Vivo Anti-Fibrotic Effects of AG1879 are Associated with Stable Changes in the Signaling/phenotype of Lung Fibroblasts

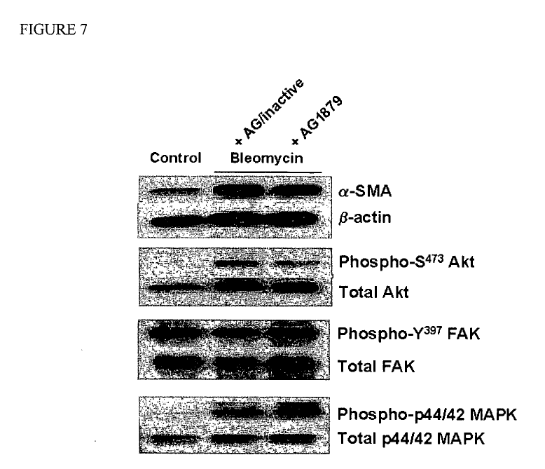

Evolving concepts on the pathogenesis of IPF suggest that alteration of fibroblast phenotypes rather than inflammation per se may play more important roles in disease pathogenesis (see, e.g., Selman, M., et al., Ann Intern Med 134:136-151 (2001); herein incorporated by reference in its entirety). Fibroblasts from the lungs of mice were isolated and cultured by tissue explants adherent on cell culture dishes. Cell cultures were maintained for two passages to establish a relatively pure population of spindle-shaped cells that uniformly stained positive for the fibroblast marker, prolyl-4-hydroxylase (see, e.g., Konttinen, Y. T., et al., J Rheumatol 16:339-345 (1989); herein incorporated by reference in its entirety) and cell lysates obtained in absence of serum for 24 h. Bleomycin injury induced the upregulation of α-SMA expression in cultured lung fibroblasts (FIG. 7), suggesting transition to a myofibroblast phenotype; this effect is mildly diminished in mice administered active AG1879 vs. inactive drug. The most significant changes were noted in the levels of PKB/Akt phosphorylation. Fibroblasts from BL-control (inactive drug) demonstrated marked increases in PKB/Akt phosphorylation that were almost undetectable in uninjured (saline-control) mice; this upregulation was significantly inhibited in fibroblasts derived from mice receiving active drug (FIG. 7). Significant alterations in FAK phosphorylation were not observed. Increased expression of phosphorylated p44/42 MAPK was observed in fibroblasts exposed to bleomycin injury, but this was not affected by administration of active AG1879. The present invention is not limited to a particular mechanism. Indeed, an understanding of the mechanism is not necessary to practice the present invention. Nonetheless, these results suggest that, similar to fibroblasts derived from patients with IPF, stable in vitro changes in fibroblast signaling/phenotype correlate with in vivo fibrotic responses to bleomycin in murine lung. The in vivo anti-fibrotic effects of the PKI, AG1879, are associated with fibroblasts that demonstrate significant reductions in PKB/Akt phosphorylation with lesser reductions in α-SMA expression.

### Example XX

### Systemic Administration of AG1879 Induces Stable Changes in the Profibrotic Signaling/Phenotype of Lung Fibroblasts

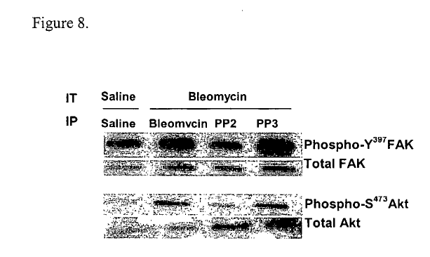

AG1879 reversed several of the phenotypic changes induced by TGF-β1 in cultured human lung fibroblasts. To determine if similar modulations occur in vivo, selected signaling pathways in fibroblasts isolated from lungs of bleomycin-injured mice were examined at day 21. Fibroblasts were initially isolated by explant culture of lung minces, expanded in 10% serum-containing medium in the absence of drugs. The cells were lysed and subjected to Western blot analysis. Differences in activation of critical protein kinases was demonstrated (See FIG. 8). The present invention is not limited to a particular mechanism. Indeed, an understanding of the mechanism is not necessary to practice the present invention. Nonetheless, these results suggest that certain phenotypic changes, which occur in cells in vivo, persist in ex vivo cell cultures. FAK and PKB/Akt activation induced in fibroblasts isolated from bleomycin-injured (fibrotic) mice were attenuated by AG1879 therapy.

### Example XXI

### In-Vivo Effects of Gleevec on Lung Fibroblast Myofibroblast Phenotype in Bleomycin-Injured Mice

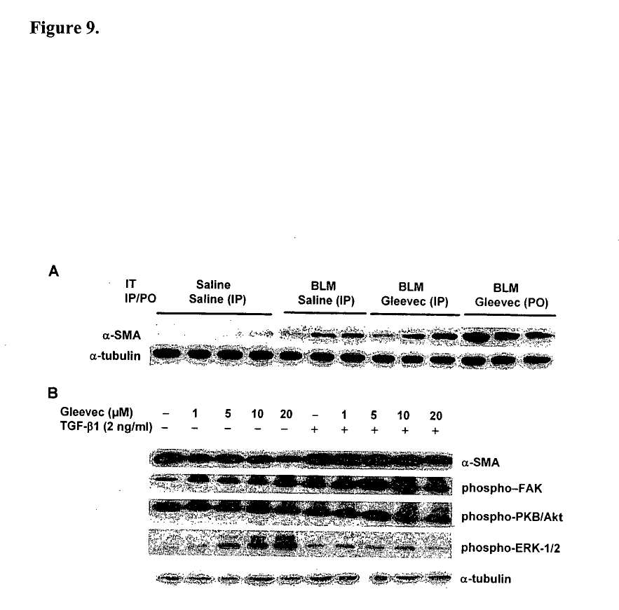

It was demonstrated that another PKI, imatinib mesylate (hereinafter, “Gleevec”), was found not to be effective as an anti-fibrotic agent. Mechanistic studies showed that lung fibroblasts cultured from Gleevec-treated mice demonstrated enhanced expression of α-SMA, a marker of myofibroblast differentiation (see FIG. 9). Additionally, study of pro-fibrotic signaling by TGF-α1 in human lung fibroblasts showed that Gleevec does not inhibit TGF-α1-induced α-SMA expression despite modest reductions in baseline expression (see FIG. 9). Gleevec promoted/enhanced TGF-α1-induced phosphorylation/activation of PKB/Akt and FAK and dose-dependently enhanced baseline phosphorylation of ERK-1/2 MAPK (see FIG. 9). The present invention is not limited to a particular mechanism. Indeed, an understanding of the mechanism is not necessary to practice the present invention. Nonetheless, these findings suggest that modulation of myofibroblast differentiation and/or survival is important for successful antifibrotic PKI therapy. Furthermore, these observations emphasize the importance of defining the modulation of fibroblast phenotypes/signaling both in vitro and in vivo.

### Example XXII

### Effects of the PKB/Akt Inhibitor, NL-71-101, on Bleomycin-Injured Pulmonary Fibrosis

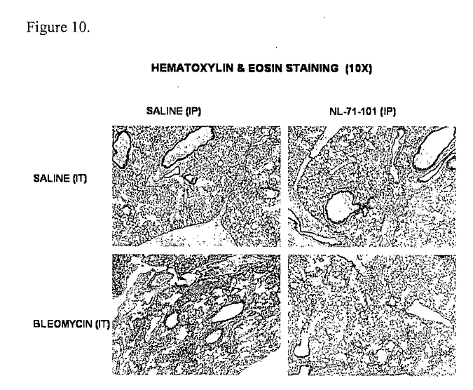

Based on observations of a important role of PKB/Akt in fibroblast/myofibroblast survival in vitro and the in vivo anti-fibrotic effects of a non-selective PKB/Akt inhibitor, AG1879, the efficacy of a more selective PKB/Akt inhibitor, NL-71-101, on bleomycin-induced pulmonary fibrosis was studied. At a dose of 10 mg/kg, the NL-71-101 PKI was well tolerated by mice and lung histology showed protection from fibrosis (see FIG. 10). This anti-fibrotic effect was observed even with the drug being administered 7 days after initial bleomycin injury.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of treating a fibrotic disorder, comprising:
a. providing:
i. a subject having a fibrous disease; and
ii. an effective amount of at least one pyrazolopyrimidine compound; and
b. administering said effective amount of at least one pyrazolopyrimidine compound to said subject.

2. The method of claim 1, wherein said fibrous disease is characteristic of activated PKB/Akt.

3. The method of claim 1, wherein said subject has been diagnosed to have elevated levels of activated PKB/Akt.

4. The method of claim 1, wherein said administering attenuates a fibrotic response.

5. The method of claim 4, wherein said fibrotic response results in fibrotic pulmonary lesions.

6. The method of claim 5, wherein said fibrotic pulmonary lesions comprise idiopathic pulmonary fibrosis (IPF).

7. The method of claim 1, wherein said at least one pyrazolopyrimidine compound comprises 4-amino-5-(4-chlorophenyl)-7-(t-butyl)pyrazolo[3,4-d]pyrimidine.

8. A therapeutic composition comprising 4-amino-5-(4-chlorophenyl)-7-(t-butyl)pyrazolo[3,4-d]pyrimidine, a second compound that targets fibrotic disorders, and a therapeutic excipient.

9. The therapeutic composition of claim 8, wherein said second compound is selected from the group consisting of: an anti-fibrotic agent, an antiviral cytokine agent, an immunosuppressive agent, and a glucocorticoid agent.

10. The therapeutic composition of claim 8, wherein said second compound is NL-71-101.

11. The therapeutic composition of claim 8, wherein said 4-amino-5-(4-chlorophenyl)-7-(t-butyl)pyrazolo[3,4-d]pyrimidine, said second compound, and said therapeutic excipient treat fibrous diseases.

12. The therapeutic composition of claim 11, wherein said fibrosis disease comprises idiopathic pulmonary fibrosis (IPF).

13. The therapeutic composition of claim 8, further comprising instructions of use of said therapeutic composition in the treatment of fibrous diseases.
